(19) United States
(12) Patent Application Publication
(10) Pub. No.: US 2026/0041570 A1
(43) Pub. Date: Feb. 12, 2026

(54) STENT, SYSTEM, AND METHOD FOR SUPPORTING A SIDE BRANCH OF A BODY LUMEN AT A BIFURCATION

(71) Applicant: BOSTON SCIENTIFIC SCIMED, INC., Maple Grove, MN (US)

(72) Inventors: John Kilcooley, Gort (IE); Javier Palomar-Moreno, Galway (IE); Michelle Hannon, Galway (IE)

(73) Assignee: BOSTON SCIENTIFIC SCIMED, INC., Maple Grove, MN (US)

(21) Appl. No.: 19/292,012

(22) Filed: Aug. 6, 2025

Related U.S. Application Data
(60) Provisional application No. 63/680,250, filed on Aug. 7, 2024.

(51) Int. Cl. A61F 2/915; A61F 2/82; A61F 2/954; A61F 2/958

(52) U.S. Cl. CPC A61F 2/915; A61F 2/954; A61F 2/958; A61F 2002/826; A61F 2230/0006

(57) ABSTRACT

A side branch stent includes a main body portion, an extension portion extending from the main body portion circumferentially around less than a full circumference of the main body portion, and an eyelet extending from the main body portion. A stent system includes a main branch stent and a side branch stent configured to engage a side wall of the main branch stent such that the main branch stent supports a main branch and the side branch stent supports a side branch. A method includes positioning first and second guidewires in side and main branches of a body lumen, advancing the side branch stent over the first guidewire into the side branch while the second guidewire is positioned through the eyelet and extends into the main branch distal of the bifurcation, and deploying the side branch stent in the side branch with the extension portion extending into the body lumen.

### CROSS REFERENCE TO RELATED APPLICATIONS

The present application claims the benefit of U.S. Provisional Patent Application Ser. No. 63/680,250, filed Aug. 7, 2024, the disclosure of which is incorporated herein by reference.

### TECHNICAL FIELD

The present disclosure pertains to medical devices, methods for manufacturing medical devices, and uses thereof. More particularly, the present disclosure pertains to a stent or endoprosthesis for implantation in a body lumen, and associated methods.

### BACKGROUND

A stent or endoprosthesis may be used in the treatment of body lumens. A stent is a generally longitudinal tubular device formed of biocompatible material which is useful to open and support various lumens in the body. For example, stents may be used in the vascular system, urogenital tract, gastrointestinal tract, esophageal tract, renal tract, tracheal/bronchial tubes, and bile duct, as well as in a variety of other applications in the body.

For treatments where a two-stent strategy is needed at a bifurcation, the double-kiss (DK) crush technique has been developed. The DK crush technique may reduce side branch restenosis by providing better side branch coverage, but there may be consequences with its use, such as (but not limited to) three layers of stent scaffold positioned against a vessel wall, which may negatively affect endothelization, etc. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, delivery method, and use alternatives for medical devices, including stent and stent systems for bifurcated vessels. One example is a side branch stent for supporting a side branch of a body lumen at a bifurcation. The side branch stent includes a main body portion having a proximal end, a distal end, a lumen extending therethrough, and an outer surface defining a circumference. The side branch stent also includes an extension portion extending proximally from the proximal end of the main body portion. The extension portion extends circumferentially around less than a full circumference of the main body portion. The side branch stent further includes an eyelet extending proximally from the proximal end of the main body portion circumferentially opposite the extension portion.

Alternatively or additionally to any of the examples herein, in another example, the eyelet extends from the main body portion at an oblique angle relative to a central longitudinal axis of the main body portion.

Alternatively or additionally to any of the examples herein, in another example, the eyelet extends radially outward from the circumference of the main body portion.

Alternatively or additionally to any of the examples herein, in another example, the eyelet is sized and configured to slidably receive a guidewire therethrough.

Alternatively or additionally to any of the examples herein, in another example, the extension portion extends circumferentially between 135 degrees and 230 degrees around the circumference of the main body portion.

Alternatively or additionally to any of the examples herein, in another example, the extension portion extends circumferentially between 150 degrees and 210 degrees around the circumference of the main body portion.

Alternatively or additionally to any of the examples herein, in another example, the extension portion extends circumferentially 180 degrees or less around the circumference of the main body portion.

Alternatively or additionally to any of the examples herein, in another example, the extension portion comprises at least one circumferential row of undulating struts disposed proximal of the proximal end of the main body portion and extending circumferentially around less than the full circumference of the main body portion.

Another example is a stent system for supporting a main branch and a side branch of a body lumen at a bifurcation, the stent system includes a main branch stent and a side branch stent. The side branch stent includes a main body portion having a proximal end, a distal end, a lumen extending therethrough, and an outer surface defining a circumference, an extension portion extending proximally from the proximal end of the main body portion circumferentially around less than a full circumference of the main body portion, and an eyelet extending proximally from the proximal end of the main body portion circumferentially opposite the extension portion. The side branch stent is configured to engage with a side wall of the main branch stent such that the main branch stent supports the main branch and the side branch stent supports the side branch of the body lumen at the bifurcation.

Alternatively or additionally to any of the examples herein, in another example, the proximal end of the main body portion is configured to engage with the side wall of the main branch stent.

Alternatively or additionally to any of the examples herein, in another example, the extension portion is configured to engage with and overlap a portion of the side wall of the main branch stent when the side branch stent is engaged with the side wall of the main branch stent.

Alternatively or additionally to any of the examples herein, in another example, the main branch stent is formed from a first layer of stent scaffold and the side branch stent is formed from a second layer of stent scaffold, and no more than two layers of stent scaffold overlap at any given location along inner walls of the main branch, the side branch, and the body lumen.

Another example is a method of supporting a body lumen at a bifurcation. The method includes positioning a first guidewire extending within the body lumen into a side branch of the body lumen at the bifurcation; positioning a second guidewire extending within the body lumen across the bifurcation into a main branch of the body lumen distal of the bifurcation; advancing a side branch stent over the first guidewire into the side branch of the body lumen adjacent the bifurcation on a first delivery balloon in a collapsed configuration, the side branch stent comprising a main body portion having a proximal end, a distal end, a lumen extending therethrough, and an outer surface defining a circumference, an extension portion extending proximally from the proximal end of the main body portion circumferentially around less than a full circumference of the main body portion, and an eyelet extending proximally from the proximal end of the main body portion circumferentially opposite the extension portion; wherein the first delivery balloon is advanced over the first guidewire into the side branch while the second guidewire is positioned through the eyelet and extends into the main branch distal of the bifurcation; and expanding the first delivery balloon within the side branch to shift the side branch stent to a deployed configuration, wherein the side branch stent engages an inner wall of the side branch and the extension portion extends into the body lumen in the deployed configuration.

Alternatively or additionally to any of the examples herein, in another example, advancing the side branch stent over the first guidewire into the side branch of the body lumen adjacent the bifurcation on the first delivery balloon in the collapsed configuration self-orients the side branch stent such that the eyelet is disposed immediately adjacent the main branch at the bifurcation and the extension portion is disposed substantially opposite the main branch adjacent the body lumen.

Alternatively or additionally to any of the examples herein, in another example, the method further includes: after shifting the side branch stent to the deployed configuration, deploying a main branch stent within the body lumen and the main branch across the bifurcation by expanding a second delivery balloon advanced over the second guidewire.

Alternatively or additionally to any of the examples herein, in another example, deploying the main branch stent includes retracting the second guidewire out of the eyelet; thereafter, advancing the second guidewire into the main branch distal of the bifurcation outside of the eyelet; advancing the main branch stent partially into the main branch on the second delivery balloon in a collapsed configuration over the second guidewire such that the main branch stent extends from the body lumen across the bifurcation into the main branch; and expanding the second delivery balloon to shift the main branch stent to a deployed configuration, wherein in the deployed configuration the main branch stent engages an inner wall of the main branch and an inner wall of the body lumen.

Alternatively or additionally to any of the examples herein, in another example, in the deployed configuration of the main branch stent and the deployed configuration of the side branch stent, a convex outer surface of the main branch stent is engaged with a concave inner surface of the extension portion of the side branch stent.

Alternatively or additionally to any of the examples herein, in another example, the method further includes: after deploying the main branch stent, advancing a side branch balloon through a side wall of the main branch stent into the lumen of the side branch stent in a deflated configuration such that a proximal portion of the side branch balloon is disposed within the main branch stent and a distal portion of the side branch balloon is disposed within the side branch stent, and inflating the side branch balloon to an inflated configuration thereby creating a lumen opening through the side wall of the main branch stent into the lumen of the side branch stent.

Alternatively or additionally to any of the examples herein, in another example, the method further includes: after expanding the side branch balloon to create the lumen opening through the side wall of the main branch stent into the lumen of the side branch stent, no more than a single layer of stent scaffold of the main branch stent and a single layer of stent scaffold of the side branch stent overlap in intimate contact with each other.

Alternatively or additionally to any of the examples herein, in another example, the method further includes: prior to deploying the side branch stent, deflecting the eyelet radially outward away from a central longitudinal axis of the main body portion such that the eyelet extends radially outward of the main body portion.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a side branch stent for supporting a side branch of a body lumen at a bifurcation;

FIG. 1A is a cross-sectional view of a portion of the side branch stent of FIG. 1;

FIG. 2 is a flat pattern view illustrating selected aspects the side branch stent of FIG. 1;

FIG. 3 is a partial side view schematically illustrating selected aspects of the side branch stent of FIG. 1;

FIG. 4 schematically illustrates selected aspects of a stent system including the side branch stent of FIG. 1;

FIGS. 5-7 illustrate selected aspects of the stent system of FIG. 4; and

FIGS. 8-22 schematically illustrate selected aspects of a system and method for supporting a side branch of a body lumen at a bifurcation.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

The disclosure describes aspects of a side branch stent 100, a stent system including the side branch stent 100, and/or a method or methods using the side branch stent 100 and/or the stent system that may be configured to be positioned in a body lumen for a variety of medical applications. For example, the side branch stent 100 may be used to treat a stenosis in a blood vessel, may be used to maintain a fluid opening or pathway in the vascular, urinary, biliary, tracheobronchial, esophageal, gastrointestinal, or renal tracts, or in some alternative configurations may be used to position a device such as an artificial valve or a filter within a body lumen. In some instances, the side branch stent 100 may be a prosthetic graft, a stent-graft, or a stent (e.g., a vascular stent, tracheal stent, bronchial stent, esophageal stent, gastrointestinal stent, biliary stent, etc.). For the purposes of this disclosure, the terms “stent” and “endoprosthesis” may generally be used interchangeably. The side branch stent 100 may be introduced endoscopically, subcutaneously, percutaneously, or surgically to be positioned within an organ, tissue, or lumen, such as a heart, artery, vein, urethra, esophagus, trachea, bronchus, bile duct, or the like.

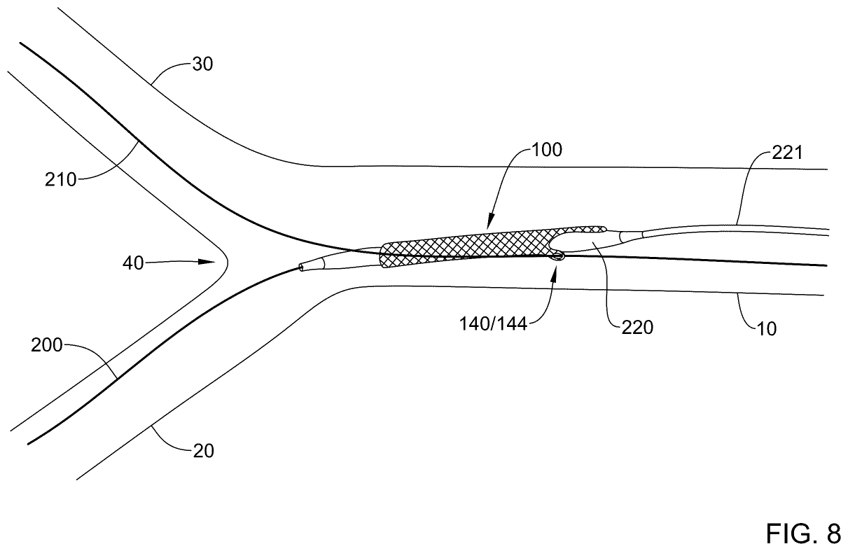

For the purpose of illustration only, the side branch stent 100 is described in the context of treating and/or supporting a side branch 20 (e.g., FIGS. 8-22) of a body lumen 10 (e.g., FIGS. 8-22), such as at a bifurcation 40 (e.g., FIGS. 8-22). However, the skilled artisan will recognize that the side branch stent 100 may have other uses and/or may be used to treat other body lumens and/or locations with little or no change to the disclosed structure(s).

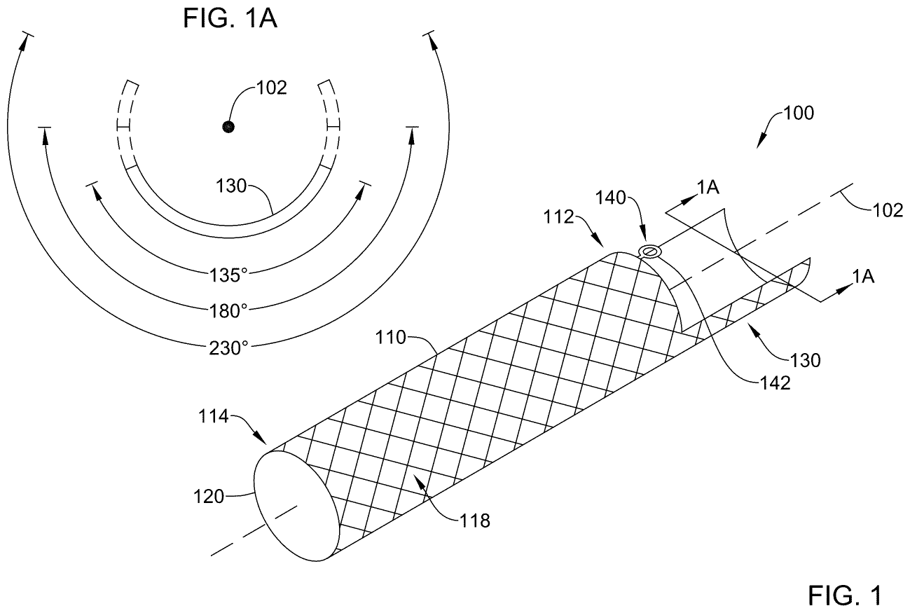

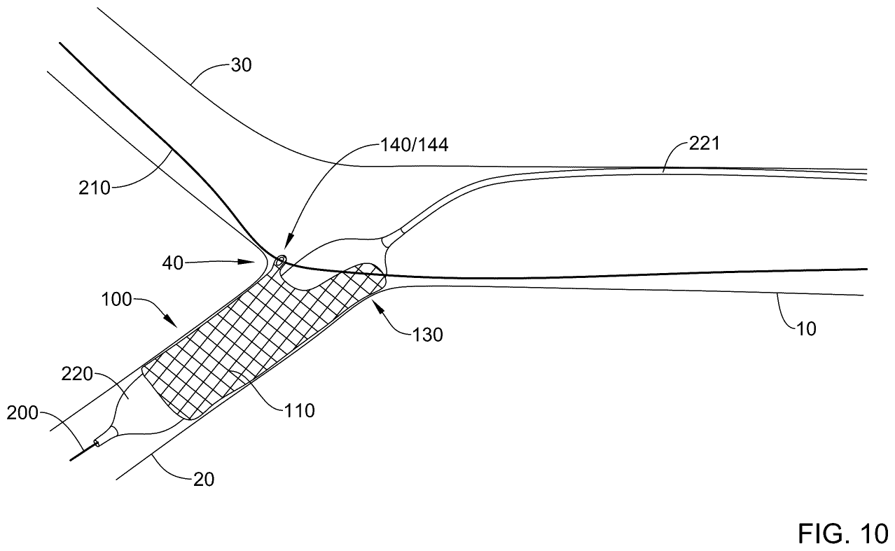

FIGS. 1-3 illustrate selected aspects of the side branch stent 100 for supporting the side branch 20 of the body lumen 10 at the bifurcation 40. The side branch stent 100 may be configured to shift from a collapsed configuration (e.g., FIG. 8) to a deployed configuration (e.g., FIG. 10). The side branch stent 100 may comprise a main body portion 110 having a proximal end 112, a distal end 114, a lumen extending therethrough along a central longitudinal axis 102 of the side branch stent 100, and an outer surface 118 defining a circumference 120 and/or an outer extent (e.g., a radial outer extent, etc.). In at least some embodiments, the outer surface 118 of the side branch stent 100 may be a convex outer surface.

The side branch stent 100 may comprise an extension portion 130 extending proximally from the proximal end 112 of the main body portion 110. In some embodiments, the extension portion 130 may extend circumferentially around less than a full circumference of the main body portion 110. In some embodiments, as seen in FIGS. 1 and 1A, the extension portion 130 may extend circumferentially between about 135 degrees and about 230 degrees around the central longitudinal axis 102, the circumference 120, and/or the outer extent of the main body portion 110. In some embodiments, the extension portion 130 may extend circumferentially between about 150 degrees and about 210 degrees around the central longitudinal axis 102, the circumference 120, and/or the outer extent of the main body portion 110. In some embodiments, the extension portion 130 may extend circumferentially about 180 degrees around the central longitudinal axis 102, the circumference 120, and/or the outer extent of the main body portion 110. In some embodiments, the extension portion 130 may extend circumferentially 180 degrees or less around the central longitudinal axis 102, the circumference 120, and/or the outer extent of the main body portion 110. Other configurations are also contemplated, including subsets thereof. In at least some embodiments, the extension portion 130 may comprise and/or may form a convex outer surface (e.g., facing away from the central longitudinal axis 102 of the main body portion 110) and a concave inner surface (e.g., facing towards the central longitudinal axis 102 of the main body portion 110).

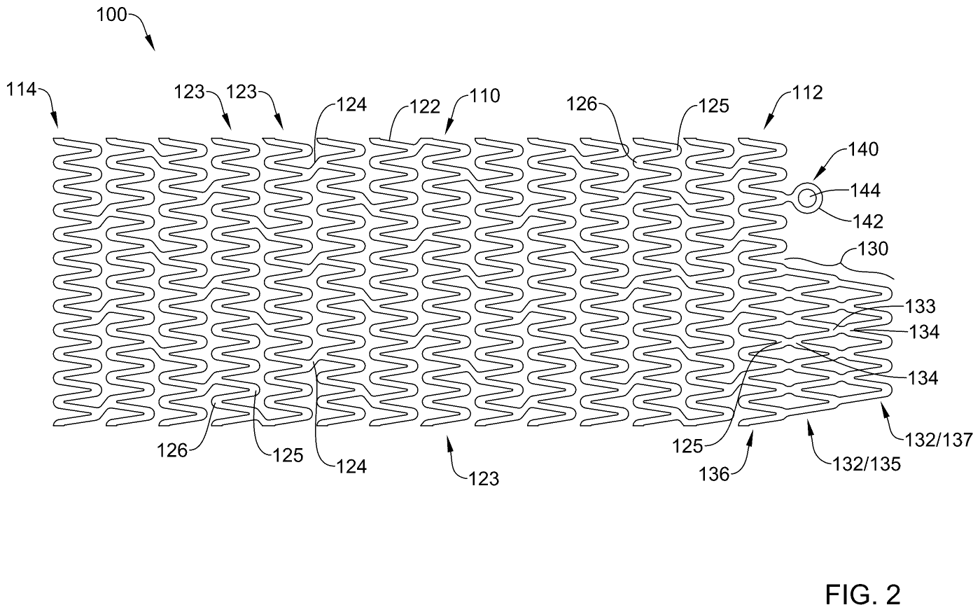

The side branch stent 100 may comprise an eyelet 140 extending proximally from the proximal end 112 of the main body portion 110 circumferentially opposite the extension portion 130, as seen in FIG. 1. The eyelet 140 may comprise a closed outer perimeter 142 and an aperture 144 extending therethrough, as seen in FIG. 2. In at least some embodiments, the eyelet 140 and/or the aperture 144 may be sized and configured to slidably receive a guidewire therethrough and/or therein. In some embodiments, the eyelet 140 may be configured to cooperate with a guidewire extending therethrough to self-orient the side branch stent 100 relative to the bifurcation 40 as the side branch stent 100 is advanced into the side branch 20 of the body lumen 10.

FIG. 2 is flat pattern view illustrating selected aspects and/or details of the side branch stent 100 in the collapsed configuration. In some embodiments, the main body portion 110 may comprise a plurality of undulating struts 122 (e.g., FIG. 2) extending circumferentially around the circumference 120. In some embodiments, the plurality of undulating struts 122 may be arranged in circumferential rows 123. In some embodiments, the main body portion 110 may comprise a plurality of longitudinal connectors 124 (e.g., FIG. 2) coupling and/or fixedly attaching longitudinally and/or axially adjacent circumferential rows 123 together. In some embodiments, two or more longitudinal connectors of the plurality of longitudinal connectors 124 may couple and/or fixedly attach longitudinally and/or axially immediately adjacent pairs of circumferential rows 123 together. Other configurations are also contemplated.

In some embodiments, the plurality of undulating struts 122 may form a sinusoidal and/or a zigzag pattern with peaks 125 and valleys 126 offset from each other in a longitudinal and/or axial direction, as seen in FIG. 2. In some embodiments, the plurality of longitudinal connectors 124 may be coupled and/or fixedly attached directly to peaks 125 of one circumferential row and valleys 126 of an immediately adjacent circumferential row. Other configurations are also contemplated.

In some embodiments, the extension portion 130 may comprise at least one circumferential row 132 of undulating struts disposed proximal of the proximal end 112 of the main body portion 110, as seen in FIG. 2. The at least one circumferential row 132 of undulating struts may form a sinusoidal and/or a zigzag pattern with peaks 133 and valleys 134 offset from each other in a longitudinal and/or axial direction. The at least one circumferential row 132 of undulating struts may extend circumferentially around less than the full circumference of the main body portion 110, as seen in FIG. 1. In some embodiments, the at least one circumferential row 132 of undulating struts may comprise two or more circumferential rows of undulating struts. In one non-limiting example, the at least one circumferential row 132 of undulating struts may comprise exactly two circumferential rows of undulating struts. Other configurations are also contemplated.

In some embodiments, the at least one circumferential row 132 of undulating struts may comprise a first circumferential row 135 of undulating struts disposed immediately adjacent the proximal end 112 of the main body portion 110. In some embodiments, each valley 134 of the first circumferential row 135 of undulating struts may be coupled and/or fixedly attached directly to an immediately adjacent peak 125 of a proximalmost circumferential row 136 of undulating struts of the main body portion 110. In some embodiments, each peak 133 of the first circumferential row 135 of undulating struts may be coupled and/or fixedly attached directly to an immediately adjacent valley 134 of an immediately adjacent circumferential row 137 of undulating struts of the extension portion 130. In some embodiments, less than every peak 133 (e.g., only some peaks 133) of the first circumferential row 135 of undulating struts may be coupled and/or fixedly attached directly to an immediately adjacent valley 134 of the immediately adjacent circumferential row 137 of undulating struts of the extension portion 130. In some embodiments, less than every valley 134 (e.g., only some valleys 134) of the first circumferential row 135 of undulating struts may be coupled and/or fixedly attached directly to an immediately adjacent peak 125 of the proximalmost circumferential row 136 of undulating struts of the main body portion 110. Other configurations are also contemplated.

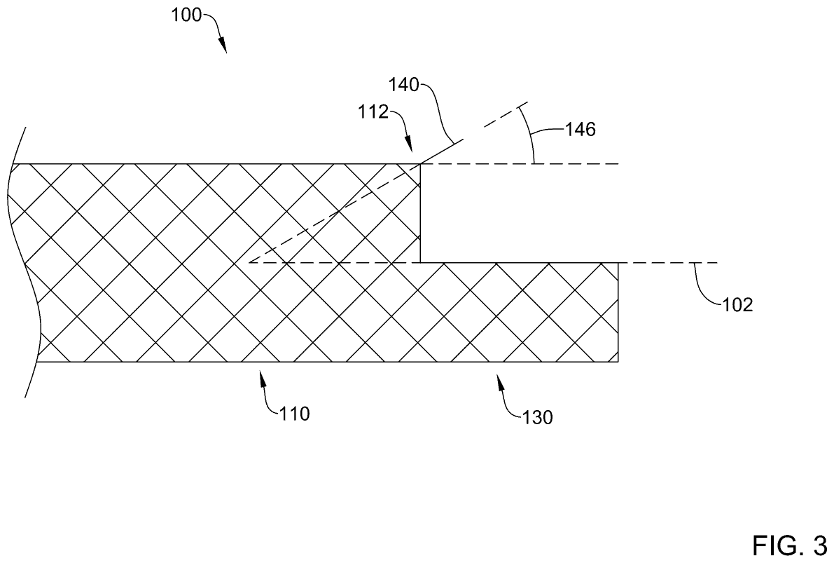

In some embodiments, the eyelet 140 may be configured to extend from the main body portion 110 at an oblique angle 146 relative to the central longitudinal axis 102 of the main body portion 110, the outer surface 118, and/or the outer extent of the main body portion 110, as seen in FIG. 3. In some embodiments, the oblique angle may be between about 30 degrees and about 75 degrees relative to the central longitudinal axis 102 of the main body portion 110, the outer surface 118, and/or the outer extent of the main body portion 110. In some embodiments, the oblique angle may be about 40, about 45, about 50, about 55, about 60, about 65, etc. degrees relative to the central longitudinal axis 102 of the main body portion 110, the outer surface 118, and/or the outer extent of the main body portion 110.

In some embodiments, the eyelet 140 may be configured to extend radially outward from the central longitudinal axis 102, the circumference 120, the outer surface 118, and/or the outer extent of the main body portion 110. In some embodiments, the eyelet 140 may be configured to extend radially outward from the central longitudinal axis 102, the circumference 120, the outer surface 118, and/or the outer extent of the main body portion 110 at the oblique angle 146 relative to the central longitudinal axis 102 of the main body portion 110, the outer surface 118, and/or the outer extent of the main body portion 110. In some embodiments, the eyelet 140 may be configured to extend radially outward from the central longitudinal axis 102, the circumference 120, the outer surface 118, and/or the outer extent of the main body portion 110 such that a guidewire may be slidably disposed within and/or through the eyelet 140 and/or the aperture 144. In some embodiments, the eyelet 140 may be configured to extend radially outward from the central longitudinal axis 102, the circumference 120, the outer surface 118, and/or the outer extent of the main body portion 110 such that the side branch stent 100 and/or the main body portion 110 is capable of sliding and/or is permitted to slide along a guidewire disposed within and/or through the eyelet 140 and/or the aperture 144 and extending alongside the side branch stent 100 and/or the main body portion 110.

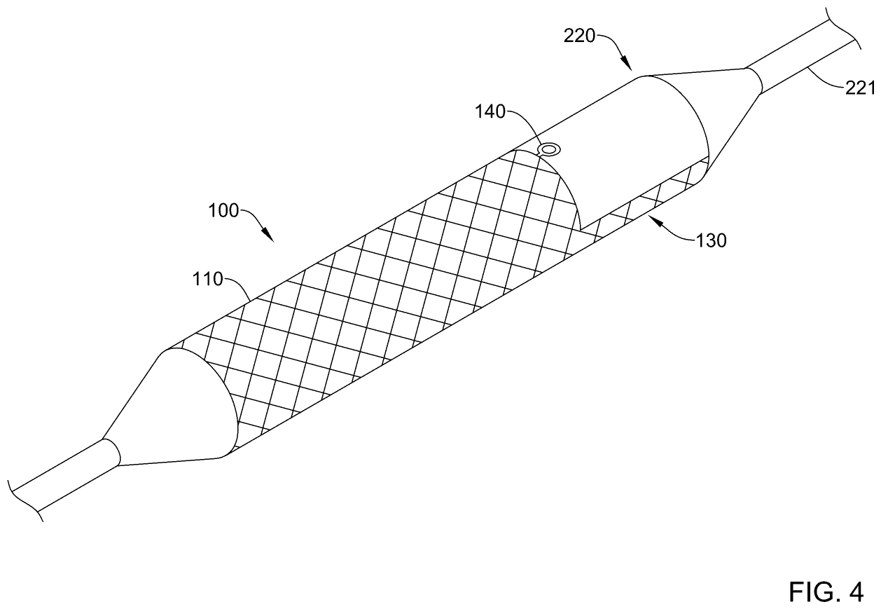

In some embodiments, a stent system may comprise the side branch stent 100. In some embodiments, the stent system may comprise a first delivery balloon 220, as seen in FIG. 4. In some embodiments, the first delivery balloon 220 may be disposed proximate a distal end of a first delivery balloon catheter 221. The first delivery balloon catheter 221 may comprise a guidewire lumen extending therethrough. The first delivery balloon catheter 221 may comprise an inflation lumen in fluid communication with an interior of the first delivery balloon 220 and fluidly couplable to a source of inflation fluid (not shown). In some embodiments, the first delivery balloon 220 may be formed from a compliant material. In some alternative configurations, the first delivery balloon 220 may be formed from a semi-compliant material or a non-compliant material. In some embodiments, the first delivery balloon 220 may preferably be formed from a polymeric material. Some suitable but non-limiting examples of polymeric materials that may be used to form the first delivery balloon 220 are discussed below. Other configurations and/or materials are also contemplated. Some additional suitable but non-limiting examples of materials that may be used to form the first delivery balloon 220, including metallic materials, composite materials, and the like, are also discussed below.

In some embodiments, the stent system may comprise the side branch stent 100 disposed on the first delivery balloon 220 in the collapsed configuration, wherein the first delivery balloon 220 is disposed in a deflated configuration. In some embodiments, the side branch stent 100 may be manufactured and/or provided with the eyelet 140 extending proximally from the proximal end 112 of the main body portion 110 substantially parallel to the central longitudinal axis 102, the circumference 120, the outer surface 118, and/or the outer extent of the main body portion 110, as seen in FIGS. 1 and 4. In such embodiments, it may be necessary to bend and/or deflect the eyelet 140 radially outward from the central longitudinal axis 102, the circumference 120, the outer surface 118, and/or the outer extent of the main body portion 110 prior to using the side branch stent 100 and/or the first delivery balloon 220 in a procedure and/or prior to advancing the side branch stent 100 and/or the first delivery balloon 220 into a body lumen, as discussed herein. In such embodiments, it may be necessary to bend and/or deflect the eyelet 140 from substantially parallel to the central longitudinal axis 102, the circumference 120, the outer surface 118, and/or the outer extent of the main body portion 110 radially outward to the oblique angle with respect to the central longitudinal axis 102, the circumference 120, the outer surface 118, and/or the outer extent of the main body portion 110 prior to using the side branch stent 100 and/or the first delivery balloon 220 in a procedure and/or prior to advancing the side branch stent 100 and/or the first delivery balloon 220 into a body lumen.

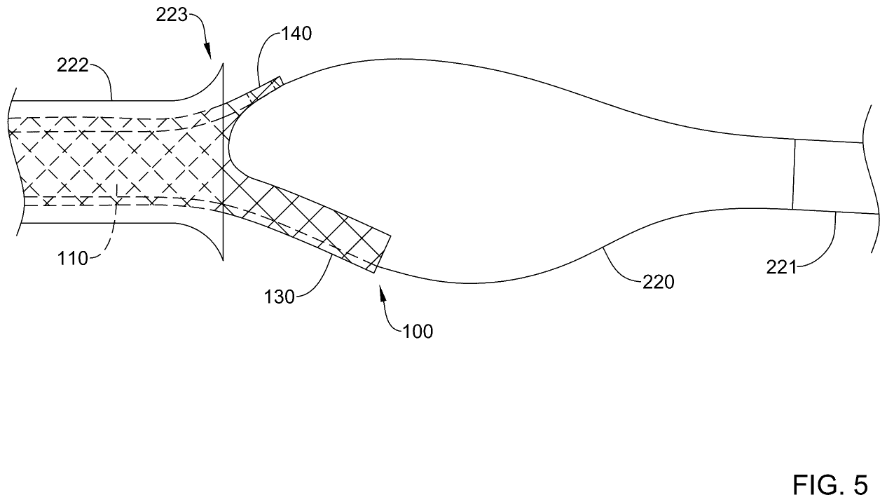

In some embodiments, the first delivery balloon 220 may be configured to cooperate with a tubular member 222 disposed over the main body portion 110 of the side branch stent 100 to bend and/or deflect the eyelet 140 radially outward from the central longitudinal axis 102, the circumference 120, the outer surface 118, and/or the outer extent of the main body portion 110, as seen in FIG. 5. In at least some embodiments, the tubular member 222 may comprise a flared end 223. In some embodiments, the flared end 223 may be shaped like the bell of a trumpet. Other configurations are also contemplated. In some embodiments, prior to use of the side branch stent 100 and/or the first delivery balloon 220, the tubular member 222 may be disposed over the main body portion 110 such that the flared end 223 of the tubular member 222 is disposed immediately adjacent the eyelet 140. An inflation fluid may be delivered to the first delivery balloon 220 to partially inflate the first delivery balloon 220 and thereby bend and/or deflect the eyelet 140 radially outward from the central longitudinal axis 102, the circumference 120, the outer surface 118, and/or the outer extent of the main body portion 110. In some embodiments, partially inflating the first delivery balloon 220 with the tubular member 222 disposed over the main body portion 110 such that the flared end 223 of the tubular member 222 is disposed immediately adjacent the eyelet 140 may bend and/or deflect the extension portion 130 radially outward from the central longitudinal axis 102, the circumference 120, the outer surface 118, and/or the outer extent of the main body portion 110 in a direction circumferentially opposite the eyelet 140. It will be appreciated that bending and/or deflecting the extension portion 130 is not required in order to use the side branch stent 100 and/or the first delivery balloon 220 and/or to advance the side branch stent 100 and/or the first delivery balloon 220 into a body lumen. After bending and/or deflecting the eyelet 140 and/or the extension portion 130 radially outward from the central longitudinal axis 102, the circumference 120, the outer surface 118, and/or the outer extent of the main body portion 110, the first delivery balloon 220 may be deflated and/or substantially returned to the deflated configuration prior to using the side branch stent 100 and/or the first delivery balloon 220 in a procedure and/or prior to advancing the side branch stent 100 and/or the first delivery balloon 220 into a body lumen.

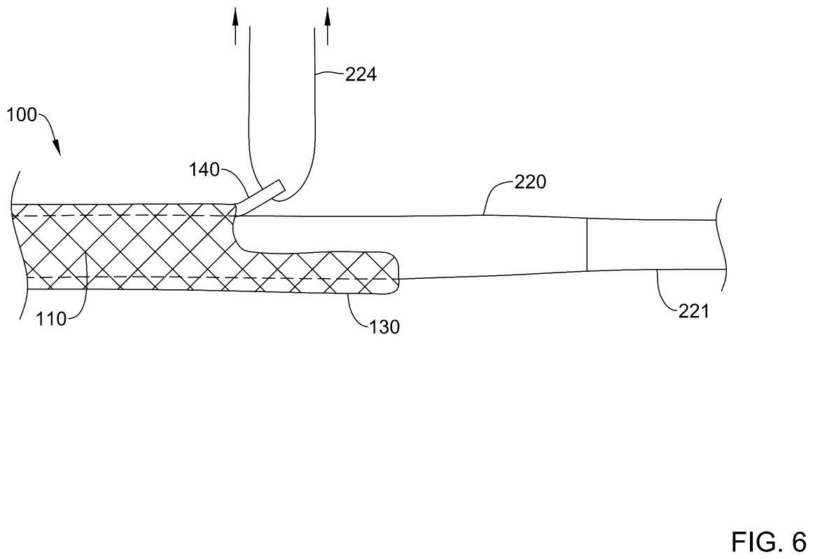

In some embodiments, the stent system may comprise a filament 224 configured to be slidably disposed through the eyelet 140 and/or the aperture 144, as seen in FIG. 6. In some embodiments, the filament 224 may be provided along with the side branch stent 100, and the filament 224 and may be inserted through the eyelet 140 and/or the aperture 144 by a user and/or attending medical personnel. In some embodiments, the stent system may be provided with the filament 224 already disposed through the eyelet 140 and/or the aperture 144. The filament 224 may be slidable within and/or through the eyelet 140 and/or the aperture 144. In some embodiments, prior to use of the side branch stent 100 and/or the first delivery balloon 220, the filament 224 may be pulled laterally away from the central longitudinal axis 102, the circumference 120, the outer surface 118, and/or the outer extent of the main body portion 110 to bend and/or deflect the eyelet 140 radially outward from the central longitudinal axis 102, the circumference 120, the outer surface 118, and/or the outer extent of the main body portion 110. After bending and/or deflecting the eyelet 140 radially outward from the central longitudinal axis 102, the circumference 120, the outer surface 118, and/or the outer extent of the main body portion 110, the filament 224 may be removed from and/or pulled through the eyelet 140 and/or the aperture 144 prior to using the side branch stent 100 and/or the first delivery balloon 220 in a procedure and/or prior to advancing the side branch stent 100 and/or the first delivery balloon 220 into a body lumen.

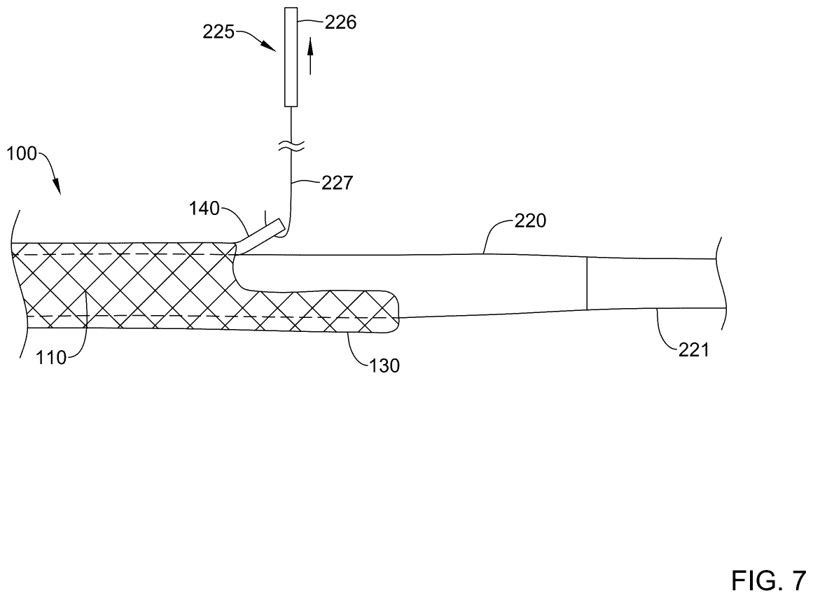

To facilitate passing a guidewire through the eyelet 140, in some embodiments, the stent system may comprise a tool 225 configured to be disposed within and/or through the eyelet 140 and/or the aperture 144, as seen in FIG. 7. It should be noted that the tool 225 is not drawn to scale. In some embodiments, the tool 225 may comprise a handle 226 and a hook element 227 extending from the handle 226. The hook element 227 may be sized and configured to be disposed within and/or through the eyelet 140 and/or the aperture 144. In some embodiments, the tool 225 may be provided along with the side branch stent 100 and the first delivery balloon 220, and the tool 225 and/or the hook element 227 may be inserted through the eyelet 140 and/or the aperture 144 by a user and/or attending medical personnel. In some embodiments, the stent system may be provided with the tool 225 and/or the hook element 227 already disposed through the eyelet 140 and/or the aperture 144. The tool 225 and/or the hook element 227 may be slidable within and/or through the eyelet 140 and/or the aperture 144. In some embodiments, prior to use of the side branch stent 100 and/or the first delivery balloon 220, the tool 225 and/or the handle 226, which may be grasped by a user and/or attending medical personnel, may be pulled laterally away from the central longitudinal axis 102, the circumference 120, the outer surface 118, and/or the outer extent of the main body portion 110 to bend and/or deflect the eyelet 140 radially outward from the central longitudinal axis 102, the circumference 120, the outer surface 118, and/or the outer extent of the main body portion 110. After bending and/or deflecting the eyelet 140 radially outward from the central longitudinal axis 102, the circumference 120, the outer surface 118, and/or the outer extent of the main body portion 110, the tool 225 and/or the hook element 227 may be removed from the eyelet 140 and/or the aperture 144 prior to using the side branch stent 100 and/or the first delivery balloon 220 in a procedure and/or prior to advancing the side branch stent 100 and/or the first delivery balloon 220 into a body lumen. In some embodiments, the stent system may be supplied with a filament (e.g., nylon) attached through the eyelet 140 which allows for bending and/or deflecting the eyelet 140 radially outward from the central longitudinal axis 102. After bending and/or deflecting the eyelet 140 radially outward, the filament may be cut and removed, to allow for subsequent advancement of the guidewire through the bent/deflected eyelet 140. Other tools and/or configurations thereof are also contemplated for bending and/or deflecting the eyelet 140.

In some embodiments, when bending and/or deflecting the eyelet 140 radially outward from the central longitudinal axis 102, the circumference 120, the outer surface 118, and/or the outer extent of the main body portion 110, the eyelet 140 may undergo at least some plastic deformation, and thus may be configured to retain its bent shape after any radially outward force applied thereto is removed.

In some embodiments, the side branch stent 100 may be formed and/or cut (e.g., via laser cutting, waterjet, machining, etc.) from a tubular member. In some embodiments, the side branch stent 100 may be formed and/or cut from a flat sheet of material and rolled into a tubular shape and/or an annular shape, where the side branch stent 100 may be permanently fixed into the tubular shape and/or the annular shape, such as by welding, bonding, etc. Other configurations are also contemplated.

In some embodiments, the side branch stent 100 may preferably be formed from a metallic material, such as stainless steel, nickel-titanium alloy(s), etc. Some suitable but non-limiting examples of metallic materials that may be used to form the side branch stent 100 are discussed below. Other configurations and/or materials are also contemplated. Some additional suitable but non-limiting examples of materials that may be used to form the side branch stent 100, including polymeric materials, composite materials, and the like, are also discussed below.

In some alternative embodiments, the side branch stent 100 may comprise a polymeric covering (not shown) coupled thereto. In some embodiments, the polymeric covering may be fixedly attached to the side branch stent 100. In some embodiments, the polymeric covering may extend along an inner surface of the side branch stent 100. In some embodiments, the polymeric covering may extend along the outer surface of the side branch stent 100. In at least some embodiments, the side branch stent 100 may be embedded within the polymeric covering. Other configurations, including combinations thereof, are also contemplated. Some suitable but non-limiting examples of polymeric materials for the polymeric covering are discussed below.

In some embodiments, the side branch stent 100 and/or the polymeric covering may include a coating disposed thereon. In some embodiments, the side branch stent 100 and/or the polymeric covering may include a lubricious coating disposed thereon. In some embodiments, the side branch stent 100 and/or the polymeric covering may include a hydrophobic coating or a hydrophilic coating disposed thereon, depending on intended use and/or situational needs. In some embodiments, the side branch stent 100 and/or the polymeric covering may be coated with a medicament or drug disposed thereon and/or may include a drug-eluting coating disposed thereon. Other configurations are also contemplated. Some suitable but non-limiting examples of coating types and/or materials that may be used with the side branch stent 100 and/or the polymeric covering are discussed below.

FIGS. 8-22 illustrate selected aspects of a stent system for supporting a side branch 20 and a main branch 30 of a body lumen 10 at a bifurcation 40, and a method of supporting the body lumen 10 at the bifurcation 40 and/or the side branch 20 and the main branch 30 of the body lumen 10 at the bifurcation 40. Prior to discussing the method itself, some aspects of the stent system are disclosed with reference to selected figures showing those aspects.

In some embodiments, the stent system may comprise a first guidewire 200 and a second guidewire 210, as seen in FIG. 8. In some embodiments, the first guidewire 200 may be configured to be positioned and/or disposed within the body lumen 10 and/or the side branch 20. In some embodiments, the second guidewire 210 may be configured to be positioned and/or disposed within the body lumen 10 and/or the main branch 30. In some embodiments, the second guidewire 210 may be configured to be positioned such that the second guidewire 210 extends into the main branch 30 distal of the bifurcation 40.

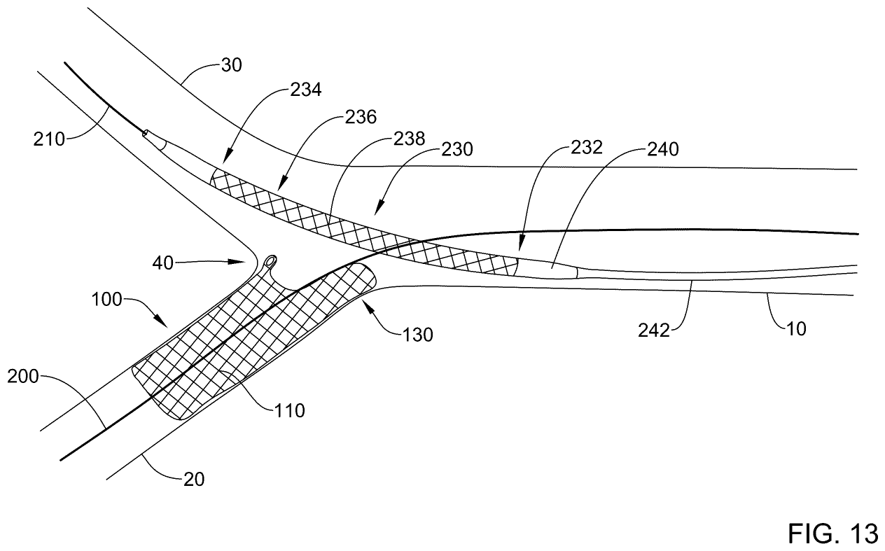

In some embodiments, the stent system may comprise a main branch stent 230 (e.g., FIG. 13). It will be appreciated that the main branch stent 230 is shown schematically for ease of understanding. However, it shall be understood that the main branch stent 230 may include and/or comprise features similar to those shown in FIG. 2 for the side branch stent 100.

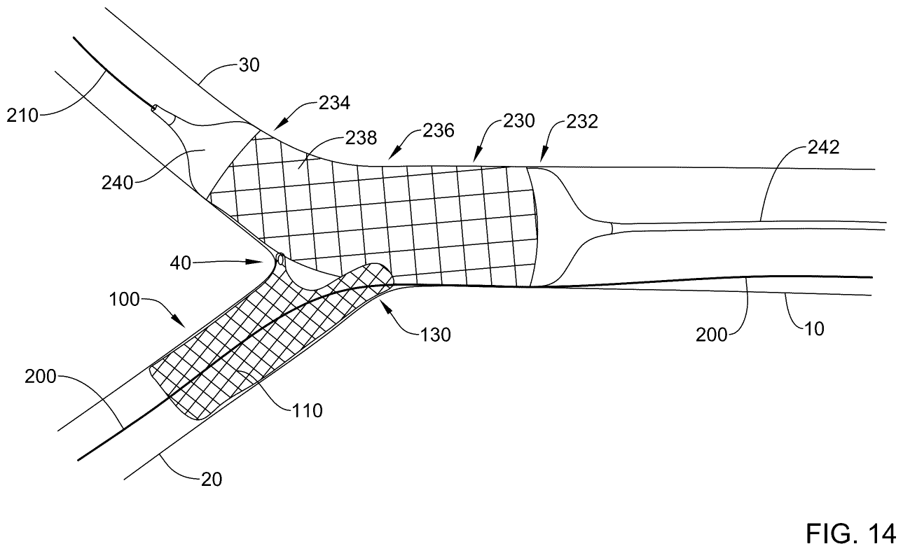

The main branch stent 230 may comprise a proximal end 232, a distal end 234, a side wall 236 extending from the proximal end 232 to the distal end 234. The side wall 236 may comprise an inner surface defining a lumen extending through the main branch stent 230 along a central longitudinal axis of the main branch stent 230. In some embodiments, the side wall 236 may comprise an outer surface 238 defining a circumference and/or an outer extent (e.g., a radial outer extent, etc.) of the main branch stent 230. The main branch stent 230 may be configured to shift from a collapsed configuration (e.g., FIG. 13) to a deployed configuration (e.g., FIG. 14). In at least some embodiments, the outer surface 238 of the main branch stent 230 may be a convex outer surface (e.g., facing away from the central longitudinal axis of the main branch stent 230).

The main branch stent 230 and/or the side wall 236 of the main branch stent 230 may comprise a plurality of undulating struts extending circumferentially around the central longitudinal axis and/or the circumference of the main branch stent 230, similar to the side branch stent 100 described above. In some embodiments, the plurality of undulating struts of the main branch stent 230 may be arranged in circumferential rows. In some embodiments, the main branch stent 230 and/or the side wall 236 of the main branch stent 230 may comprise a plurality of longitudinal connectors coupling and/or fixedly attaching longitudinally and/or axially adjacent circumferential rows of the main branch stent 230. In some embodiments, two or more longitudinal connectors of the plurality of longitudinal connectors of the main branch stent 230 may couple and/or fixedly attach longitudinally and/or axially immediately adjacent pairs of circumferential rows of the main branch stent 230 together. Other configurations are also contemplated.

In some embodiments, the plurality of undulating struts of the main branch stent 230 may form a sinusoidal and/or a zigzag pattern with peaks and valleys offset from each other in a longitudinal and/or axial direction. In some embodiments, the plurality of longitudinal connectors of the main branch stent 230 may be coupled and/or fixedly attached directly to peaks of one circumferential row and valleys of an immediately adjacent circumferential row of the main branch stent 230. Other configurations are also contemplated.

In some embodiments, the main branch stent 230 may be formed and/or cut (e.g., via laser cutting, waterjet, machining, etc.) from a tubular member. In some embodiments, the main branch stent 230 may be formed and/or cut from a flat sheet of material and rolled into a tubular shape and/or an annular shape, where the main branch stent 230 may be permanently fixed into the tubular shape and/or the annular shape, such as by welding, bonding, etc. Other configurations are also contemplated.

In some embodiments, the main branch stent 230 may preferably be formed from a metallic material, such as stainless steel, nickel-titanium alloy(s), etc. Some suitable but non-limiting examples of metallic materials that may be used to form the main branch stent 230 are discussed below. Other configurations and/or materials are also contemplated. Some additional suitable but non-limiting examples of materials that may be used to form the main branch stent 230, including polymeric materials, composite materials, and the like, are also discussed below.

In some alternative embodiments, the main branch stent 230 may comprise a polymeric covering (not shown) coupled thereto. In some embodiments, the polymeric covering may be fixedly attached to the main branch stent 230. In some embodiments, the polymeric covering may extend along an inner surface of the main branch stent 230. In some embodiments, the polymeric covering may extend along the outer surface of the main branch stent 230. In at least some embodiments, the main branch stent 230 may be embedded within the polymeric covering. Other configurations, including combinations thereof, are also contemplated. Some suitable but non-limiting examples of polymeric materials for the polymeric covering are discussed below.

In some embodiments, the main branch stent 230 and/or the polymeric covering may include a coating disposed thereon. In some embodiments, the main branch stent 230 and/or the polymeric covering may include a lubricious coating disposed thereon. In some embodiments, the main branch stent 230 and/or the polymeric covering may include a hydrophobic coating or a hydrophilic coating disposed thereon, depending on intended use and/or situational needs. In some embodiments, the main branch stent 230 and/or the polymeric covering may be coated with a medicament or drug disposed thereon and/or may include a drug-eluting coating disposed thereon. Other configurations are also contemplated. Some suitable but non-limiting examples of coating types and/or materials that may be used with the main branch stent 230 and/or the polymeric covering are discussed below.

In some embodiments, the side branch stent 100 may be configured to engage with the side wall 236 and/or the outer surface 238 of the main branch stent 230 such that the main branch stent 230 supports the main branch 30 of the body lumen 10 and the side branch stent 100 supports the side branch 20 of the body lumen 10 at the bifurcation 40, as discussed herein. In some embodiments, the proximal end 112 of the main body portion 110 of the side branch stent 100 may be configured to engage with and/or contact the side wall 236 and/or the outer surface 238 of the main branch stent 230, after the side branch stent 100 and the main branch stent 230 have been deployed within the body lumen 10, the side branch 20, and/or the main branch 30 at the bifurcation.

In some embodiments, the main branch stent 230 may be formed from a first layer of stent scaffold (e.g., the side wall 236, the plurality of undulating struts, the circumferential rows, the plurality of longitudinal connectors, etc.). In some embodiments, the side branch stent 100 may be formed from a second layer of stent scaffold (e.g., the main body portion, the plurality of undulating struts 122, the circumferential rows 123, the plurality of longitudinal connectors 124, the extension portion 130, etc.). In some embodiments, the side branch stent 100 and the main branch stent 230 may be configured such that after deployment, no more than two layers of stent scaffold overlap at any given circumferential location along inner walls of the body lumen 10, the side branch 20, and the main branch 30. For the purpose of this disclosure, the term “stent scaffold” refers only to the material forming the stent itself (e.g., the plurality of undulating struts, the plurality of longitudinal connectors, etc.) and does not include any additional layers, such as coverings and/or coatings that may be present.

In some embodiments, the stent system may comprise a second delivery balloon 240, as seen in FIG. 13. In some embodiments, the second delivery balloon 240 may be disposed proximate a distal end of a second delivery balloon catheter 242. The second delivery balloon catheter 242 may comprise a guidewire lumen extending therethrough. The second delivery balloon catheter 242 may comprise an inflation lumen in fluid communication with an interior of the second delivery balloon 240 and fluidly couplable to a source of inflation fluid (not shown). In some embodiments, the stent system may comprise the main branch stent 230 disposed on the second delivery balloon 240 in the collapsed configuration, wherein the second delivery balloon 240 is disposed in a deflated configuration. In some embodiments, the second delivery balloon 240 may be formed from a compliant material. In some alternative configurations, the second delivery balloon 240 may be formed from a semi-compliant material or a non-compliant material. In some embodiments, the second delivery balloon 240 may preferably be formed from a polymeric material. Some suitable but non-limiting examples of polymeric materials that may be used to form the second delivery balloon 240 are discussed below. Other configurations and/or materials are also contemplated. Some additional suitable but non-limiting examples of materials that may be used to form the second delivery balloon 240, including metallic materials, composite materials, and the like, are also discussed below.

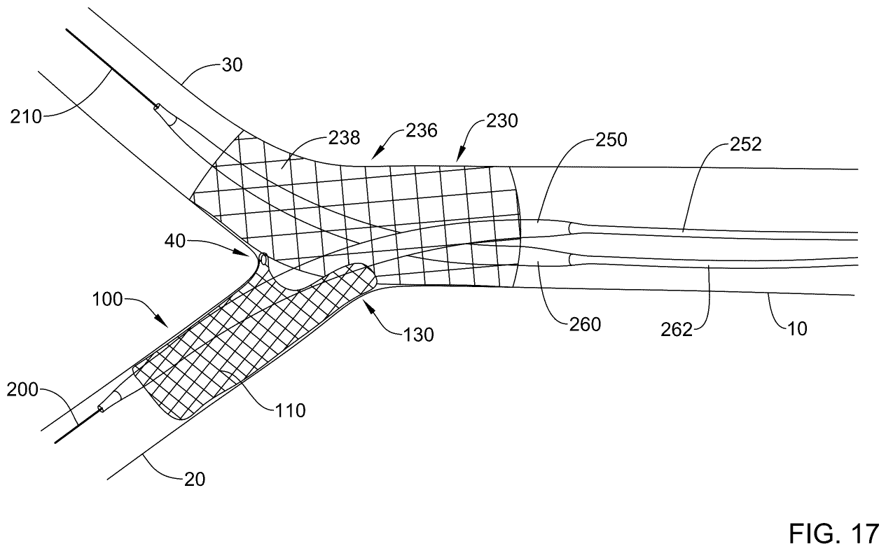

In some embodiments, the stent system may comprise a side branch balloon 250, as seen in FIG. 17. In some embodiments, the side branch balloon 250 may be disposed proximate a distal end of a side branch balloon catheter 252. The side branch balloon catheter 252 may comprise a guidewire lumen extending therethrough. The side branch balloon catheter 252 may comprise an inflation lumen in fluid communication with an interior of the side branch balloon 250 and fluidly couplable to a source of inflation fluid (not shown). In some embodiments, a main body portion of the side branch balloon 250 may have a length that is at least as long as the side branch stent 100. In some embodiments, the side branch balloon 250 may be formed from a non-compliant material. In some alternative configurations, the side branch balloon 250 may be formed from a semi-compliant material or a compliant material. In some embodiments, the side branch balloon 250 may preferably be formed from a polymeric material. Some suitable but non-limiting examples of polymeric materials that may be used to form the side branch balloon 250 are discussed below. Other configurations and/or materials are also contemplated. Some additional suitable but non-limiting examples of materials that may be used to form the side branch balloon 250, including metallic materials, composite materials, and the like, are also discussed below.

In some embodiments, the stent system may comprise a main branch balloon 260, as seen in FIG. 17. In some embodiments, the main branch balloon 260 may be disposed proximate a distal end of a main branch balloon catheter 262. The main branch balloon catheter 262 may comprise a guidewire lumen extending therethrough. The main branch balloon catheter 262 may comprise an inflation lumen in fluid communication with an interior of the main branch balloon 260 and fluidly couplable to a source of inflation fluid (not shown). In some embodiments, a main body portion of the main branch balloon 260 may have a length that is at least as long as the main branch stent 230. In some embodiments, the main branch balloon 260 may be formed from a non-compliant material. In some alternative configurations, the main branch balloon 260 may be formed from a semi-compliant material or a compliant material. In some embodiments, the main branch balloon 260 may preferably be formed from a polymeric material. Some suitable but non-limiting examples of polymeric materials that may be used to form the main branch balloon 260 are discussed below. Other configurations and/or materials are also contemplated. Some additional suitable but non-limiting examples of materials that may be used to form the main branch balloon 260, including metallic materials, composite materials, and the like, are also discussed below.

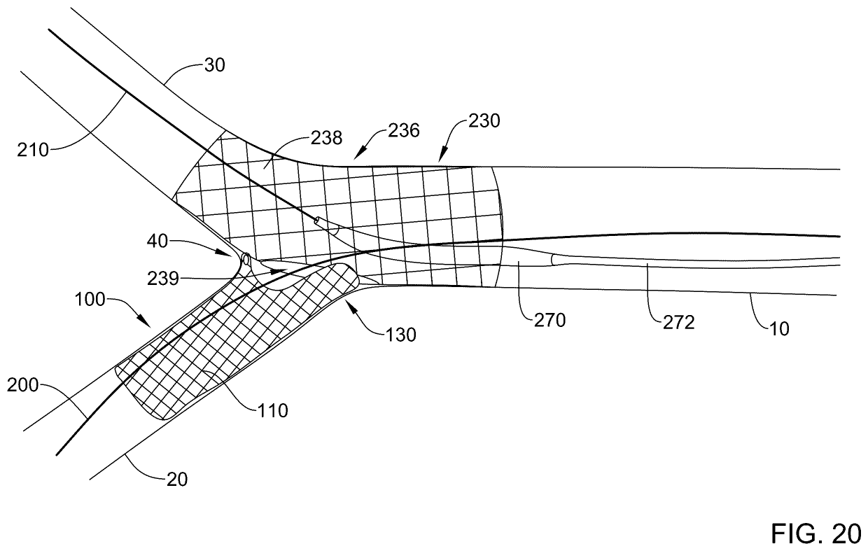

In some embodiments, the stent system may comprise a Proximal Optimization Technique (POT) balloon 270, as seen in FIG. 20, to post-dilate the proximal end of the stent and correct malapposition against the vessel wall, since in tapered vessels the stent is commonly sized for the narrower distal end and overexpanded with a shorter balloon (e.g., POT balloon) in the proximal end. In some embodiments, the POT balloon 270 may be disposed proximate a distal end of a POT balloon catheter 272. The POT balloon catheter 272 may comprise a guidewire lumen extending therethrough. The POT balloon catheter 272 may comprise an inflation lumen in fluid communication with an interior of the POT balloon 270 and fluidly couplable to a source of inflation fluid (not shown). In some embodiments, a main body portion of the POT balloon 270 may have a length that is shorter than the main branch stent 230. In some embodiments, the POT balloon 270 may be formed from a non-compliant material. In some alternative configurations, the POT balloon 270 may be formed from a semi-compliant material or a compliant material. In some embodiments, the POT balloon 270 may preferably be formed from a polymeric material. Some suitable but non-limiting examples of polymeric materials that may be used to form the POT balloon 270 are discussed below. Other configurations and/or materials are also contemplated. Some additional suitable but non-limiting examples of materials that may be used to form the POT balloon 270, including metallic materials, composite materials, and the like, are also discussed below.

Returning now to FIG. 8, in some embodiments, the method of supporting the body lumen 10 (and/or the side branch 20 and the main branch 30 of the body lumen 10) at the bifurcation 40 may comprise positioning the first guidewire 200 extending within the body lumen 10 into the side branch 20 of the body lumen 10 at the bifurcation 40. In some embodiments, the positioning of the first guidewire 200 may comprise inserting and/or advancing the guidewire into the body lumen 10, advancing the first guidewire 200 to a position adjacent the bifurcation 40, and directing and/or advancing the first guidewire 200 into the side branch 20 of the body lumen 10.

In some embodiments, the method may comprise positioning the second guidewire 210 extending within the body lumen 10 across the bifurcation 40 into the main branch 30 of the body lumen 10 distal of the bifurcation 40. In some embodiments, the positioning of the second guidewire 210 may comprise inserting and/or advancing the guidewire into the body lumen 10, advancing the second guidewire 210 to a position adjacent the bifurcation 40, and directing and/or advancing the second guidewire 210 across the bifurcation 40 into the main branch 30 of the body lumen 10 distal of the bifurcation 40.

In some embodiments, the method may comprise loading the first delivery balloon 220 with the side branch stent 100 disposed thereon in the collapsed configuration onto the first guidewire 200, wherein the first guidewire 200 is slidably disposed within the guidewire lumen of the first delivery balloon catheter 221 and the second guidewire 210 is slidably disposed within and/or through the eyelet 140 and/or the aperture 144.

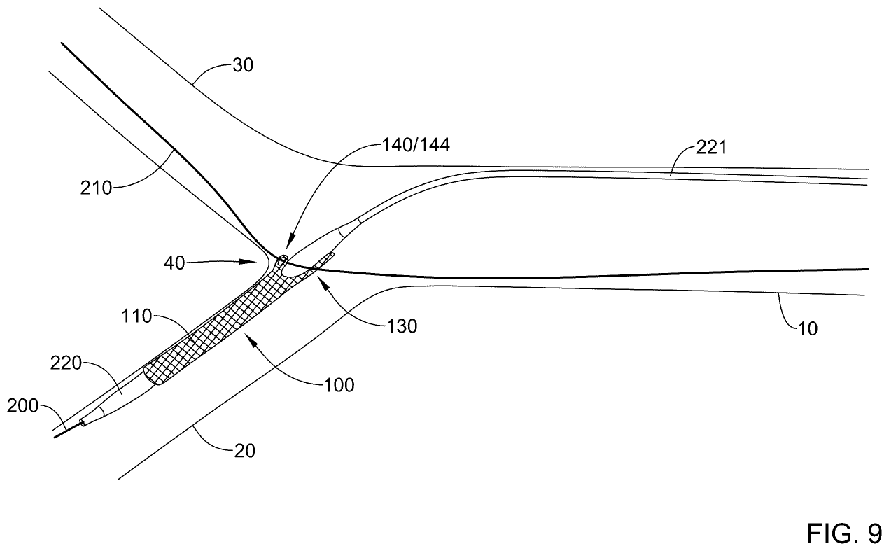

In some embodiments, the method may comprise advancing the side branch stent 100 over the first guidewire 200 into the side branch 20 of the body lumen 10 adjacent the bifurcation 40 on the first delivery balloon 220 in the collapsed configuration, as seen in FIG. 9. The side branch stent 100, the first delivery balloon 220, and/or the first delivery balloon catheter 221 may be advanced over the first guidewire 200 while the second guidewire 210 is positioned within and/or through the eyelet 140 and/or the aperture 144 and extends into the main branch 30 distal of the bifurcation 40.

In some embodiments, advancing the side branch stent 100 over the first guidewire 200 into the side branch 20 of the body lumen 10 adjacent the bifurcation 40 on the first delivery balloon 220 and/or the first delivery balloon catheter 221 in the collapsed configuration self-orients the side branch stent 100 such that the eyelet 140 is disposed immediately adjacent the main branch 30 at the bifurcation 40 and the extension portion 130 is disposed substantially opposite the main branch 30 and/or the bifurcation 40 adjacent the body lumen 10, as seen in FIG. 9. In other words, as the side branch stent 100 is advanced through the body lumen 10 with the first guidewire 200 extending through the eyelet 140, the side branch stent 100 may be self-oriented such that the eyelet 140 is oriented immediately adjacent the main branch 30 (i.e., at the apex or flow divider between the main branch 30 and the side branch 20). In some embodiments, the eyelet 140 may be configured to cooperate with the first guidewire 200 extending therethrough to self-orient the side branch stent 100 relative to the bifurcation 40 as the side branch stent 100 is advanced into the side branch 20 of the body lumen 10.

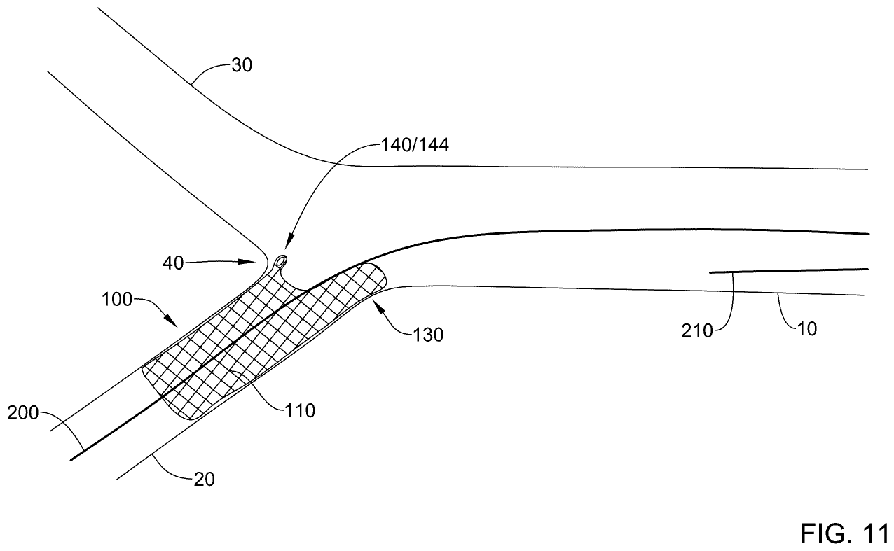

In some embodiments, the method may comprise expanding and/or inflating the first delivery balloon 220 to the inflated configuration within the side branch 20 to deploy the side branch stent 100 and/or to shift the side branch stent 100 from the collapsed configuration to the deployed configuration, wherein the outer surface 118 of the side branch stent 100 and/or the main body portion 110 engages an inner wall of the side branch 20 and the extension portion 130 extends into the body lumen 10 in the deployed configuration, as seen in FIG. 10. In some embodiments, the method may comprise collapsing and/or deflating the first delivery balloon 220 toward and/or to the deflated configuration and retracting and/or removing the first delivery balloon 220 from the side branch 20 and/or the body lumen 10 over the first guidewire 200, as seen in FIG. 11. In some embodiments, the first guidewire 200 may be held and/or may remain in place within the side branch 20 and/or the lumen of the side branch stent 100. In some embodiments, the first guidewire 200 may be removed from the side branch 20 and/or the body lumen 10 after removing the first delivery balloon 220 from the side branch 20 and/or the body lumen 10. In some alternative embodiments, the first guidewire 200 may be removed from the side branch 20 and/or the body lumen 10 concurrently with the first delivery balloon 220.

In some embodiments, the method may comprise, prior to deploying the side branch stent 100 and/or prior to advancing the side branch stent 100 over the first guidewire 200 into the side branch 20, deflecting the eyelet 140 radially outward from the central longitudinal axis 102 of the main body portion 110 such that the eyelet 140 extends radially outward of the main body portion 110, the outer surface 118 of the main body portion 110, and/or the outer extent of the main body portion 110, as discussed above and shown in FIGS. 5-7. In at least some embodiments, the side branch stent 100 may be advanced over the first guidewire 200 into the side branch 20 with the eyelet 140 extending radially outward from the central longitudinal axis 102, the circumference 120, the outer surface 118, and/or the outer extent of the main body portion 110 and the second guidewire 210 disposed within and/or extending through the eyelet 140 and/or the aperture 144.

In some embodiments, the method may comprise, after deploying the side branch stent 100 and/or after shifting the side branch stent 100 to the deployed configuration, deploying the main branch stent 230 within the body lumen 10 and the main branch 30 across the bifurcation 40 by expanding and/or inflating the second delivery balloon 240 advanced over the second guidewire 210, as seen in FIGS. 13-14.

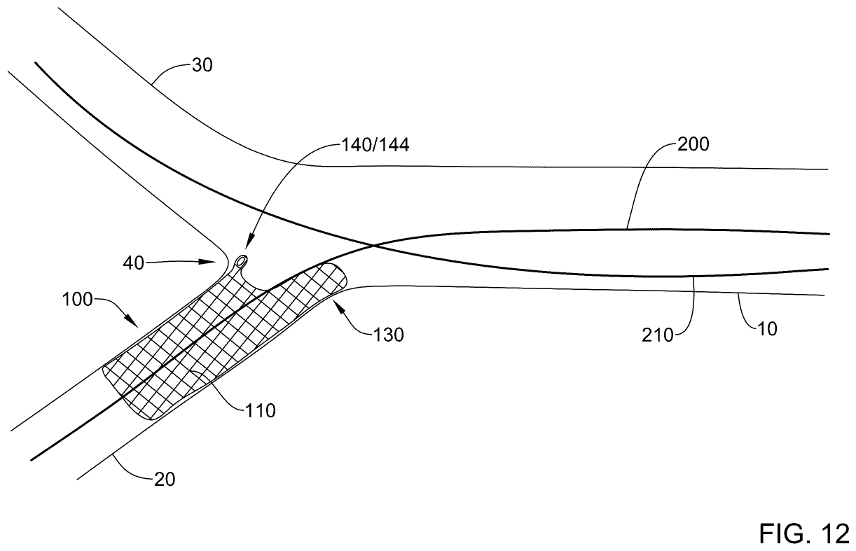

In some embodiments, deploying the main branch stent 230 within the body lumen 10 and the main branch 30 across the bifurcation 40 may comprise retracting the second guidewire 210 out of the eyelet 140 and/or the aperture 144, as seen in FIG. 11, thereafter, advancing the second guidewire 210 (or in some alternative configurations, a separate guidewire exchanged for the second guidewire 210) into the main branch 30 distal of the bifurcation 40 outside of the eyelet 140 (e.g., without passing through the eyelet 140), as seen in FIG. 12, advancing the main branch stent 230 partially into the main branch 30 on the second delivery balloon 240 in the collapsed configuration over the second guidewire 210 such that the main branch stent 230 extends from the body lumen 10 across the bifurcation 40 into the main branch 30, as seen in FIG. 13, and expanding and/or inflating the second delivery balloon 240 to the inflated configuration to shift the main branch stent 230 from the collapsed configuration to the deployed configuration, as seen in FIG. 14.

In some embodiments, expanding and/or inflating the second delivery balloon 240 to the inflated configuration, and/or deploying the main branch stent 230, may push and/or shift the first guidewire 200, when present, laterally within the body lumen 10 such that the first guidewire 200 is disposed between the main branch stent 230 and the inner wall of the body lumen 10 and/or the first guidewire 200 is urged against the inner wall of the body lumen 10, as seen in FIG. 14. In some embodiments, the first guidewire 200 may “float” within the body lumen 10 until urged against the inner wall of the body lumen 10 by the main branch stent 230.

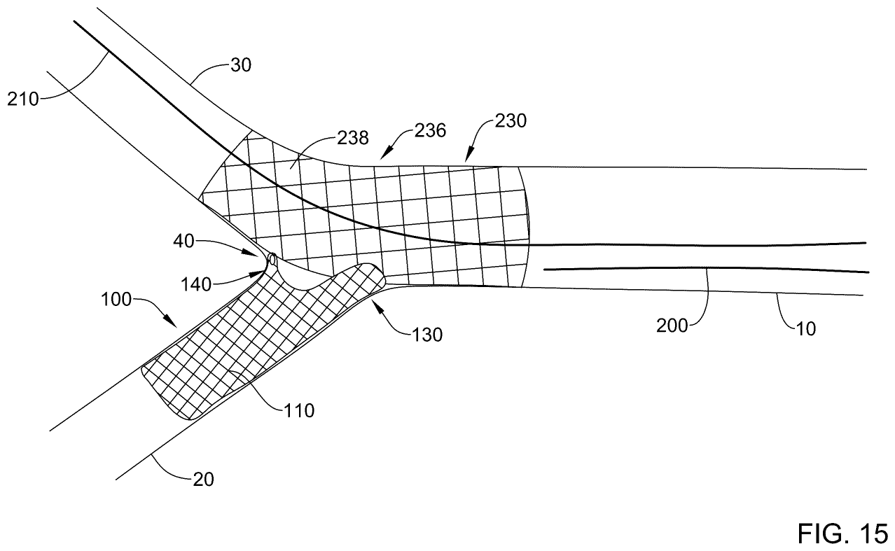

After deploying the main branch stent 230, the second delivery balloon 240 may be deflated and/or shifted toward and/or to the deflated configuration and removed from the body lumen 10, as seen in FIG. 15, thereby leaving the main branch stent 230 extending across the bifurcation 40 in the deployed configuration. Thereafter, the first guidewire 200, when present, may be retracted from the side branch 20 to a position proximal of the main branch stent 230 within the body lumen 10, as seen in FIG. 15. In some alternative embodiments, the first guidewire 200 may be removed from the body lumen 10 and exchanged with a different “first” guidewire that will take the place of the first guidewire 200 for the remainder of the method/procedure. For the purpose of illustration, the method and figures refer to the first guidewire 200 throughout, but it shall be understood that a different “first” guidewire (e.g., a replacement and/or substitute guidewire) may be used for the remainder of the method/procedure. In at least some embodiments, where a different “first” guidewire is used, physical properties of the different “first” guidewire may be the same as, similar to, or different from those of the first guidewire 200, as desired.

In some embodiments, in the deployed configuration of the main branch stent 230 and the deployed configuration of the side branch stent 100, the main branch stent 230 is configured to engage and/or engages the inner wall of the main branch 30, the inner wall of the body lumen 10, and the extension portion 130 of the side branch stent 100. In some embodiments, in the deployed configuration of the main branch stent 230 and the deployed configuration of the side branch stent 100, the outer surface 238 of the main branch stent 230 and/or the side wall 236 of the main branch stent 230 is configured to engage and/or engages the inner wall of the main branch 30 and the inner wall of the body lumen 10. In some embodiments, in the deployed configuration of the main branch stent 230 and the deployed configuration of the side branch stent 100, the outer surface 238 of the main branch stent 230 and/or the side wall 236 of the main branch stent 230 is configured to engage and/or engages the extension portion 130 of the side branch stent 100.

In some embodiments, in the deployed configuration of the main branch stent 230 and the deployed configuration of the side branch stent 100, the outer surface 238 of the main branch stent 230 and/or the side wall 236 of the main branch stent 230 is configured to engage and/or engages an inner surface or a radially inward facing surface of the extension portion 130 of the side branch stent 100. In some embodiments, in the deployed configuration the main branch stent 230 and the deployed configuration of the side branch stent 100, a convex outer surface of the main branch stent 230 and/or the side wall 236 of the main branch stent 230 is configured to engage and/or engages a concave inner surface of the extension portion 130 of the side branch stent 100.

In some embodiments, in the deployed configuration of the main branch stent 230 and the deployed configuration of the side branch stent 100, at least a portion of the proximal end 112 of the main body portion 110 of the side branch stent 100 may be configured to engage and/or engages the outer surface 238 of the main branch stent 230 and/or the side wall 236. In some embodiments, in the deployed configuration of the main branch stent 230 and the deployed configuration of the side branch stent 100, the eyelet 140 may engage with the side wall 236 of the main branch stent 230. In some embodiments, in the deployed configuration of the main branch stent 230 and the deployed configuration of the side branch stent 100, the eyelet 140 may extend through the side wall 236 of the main branch stent 230 into the lumen of the main branch stent 230.

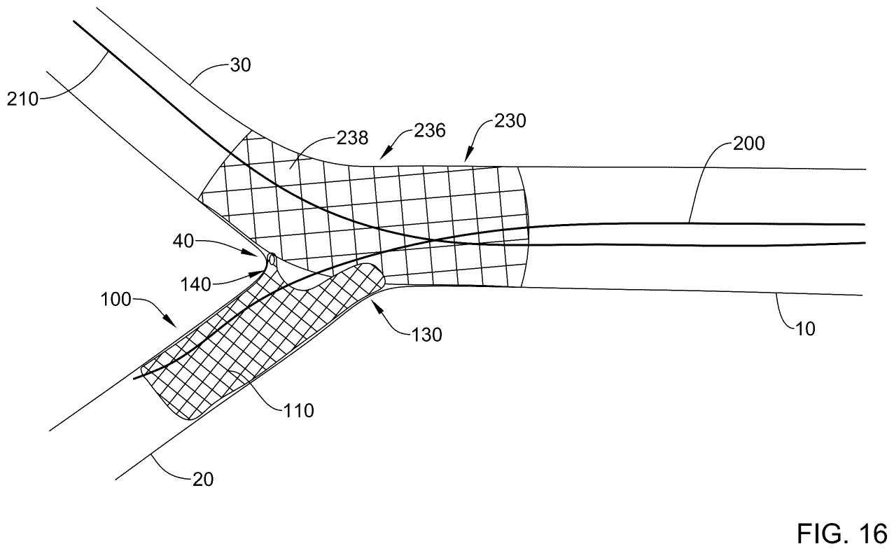

In some embodiments, the method may comprise, after deploying the main branch stent 230 and removing the second delivery balloon 240 from the body lumen 10, advancing the first guidewire 200 (or the different “first” guidewire, where present) through the side wall 236 of the main branch stent 230 into the side branch 20 and/or the lumen of the side branch stent 100, as seen in FIG. 16. In some embodiments, the first guidewire 200 may be advanced through the side wall 236 of the main branch stent 230 between two adjacent circumferential rows of the main branch stent 230.

In some embodiments, the method may comprise, after deploying the main branch stent 230 and removing the second delivery balloon 240 from the body lumen 10, advancing the side branch balloon 250 through the side wall 236 of the main branch stent 230 into the lumen of the side branch stent 100 in a deflated configuration such that a proximal portion of the side branch balloon 250 is disposed within the main branch stent 230 and/or the lumen of the main branch stent 230 and a distal portion of the side branch balloon 250 is disposed within the side branch stent 100 and/or the lumen of the side branch stent 100, as seen in FIG. 17. In at least some embodiments, the side branch balloon 250 may be advanced through the side wall 236 of the main branch stent 230 into the lumen of the side branch stent 100 in the deflated configuration over the first guidewire 200 (or the different “first” guidewire, where present).

In some embodiments, the method may comprise, after deploying the main branch stent 230 and removing the second delivery balloon 240 from the body lumen 10, advancing the main branch balloon 260 into the lumen of the main branch stent 230 in a deflated configuration, as seen in FIG. 17. In at least some embodiments, the main branch balloon 260 may be advanced into the lumen of the main branch stent 230 in the deflated configuration over the second guidewire 210.

In some embodiments, the side branch balloon 250 may be advanced into the lumen of the side branch stent 100 before the main branch balloon 260 is advanced into the lumen of the main branch stent 230. In some embodiments, the main branch balloon 260 may be advanced into the lumen of the main branch stent 230 before the side branch balloon 250 is advanced into the lumen of the side branch stent 100. In some alternative configurations, the side branch balloon 250 may be advanced into the lumen of the side branch stent 100 alongside and/or concurrently with the main branch balloon 260 being advanced into the lumen of the main branch stent 230.

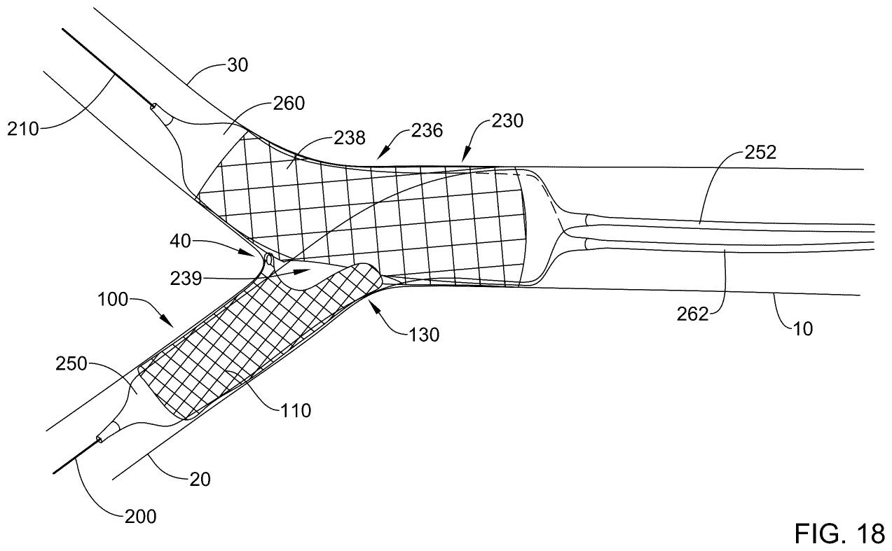

In some embodiments, the method may comprise expanding and/or inflating the side branch balloon 250 to the inflated configuration while the side branch balloon 250 is disposed through the side wall 236 of the main branch stent 230 thereby creating a lumen opening 239 through the side wall 236 of the main branch stent 230 into the lumen of the side branch stent 100, as seen in FIG. 18. In some embodiments, the lumen opening 239 has a radial extent greater than a radial extent of the side branch balloon 250 in the deflated configuration. In some embodiments, the method may comprise expanding and/or inflating the main branch balloon 260 to the inflated configuration while the side branch balloon 250 is disposed through the side wall 236 of the main branch stent 230 and inflated, as seen in FIG. 18.

In some embodiments, the side branch balloon 250 and the main branch balloon 260 may be expanded and/or inflated sequentially. For example, the side branch balloon 250 may be expanded and/or inflated before the main branch balloon 260, or the main branch balloon 260 may be expanded and/or inflated before the side branch balloon 250. In some embodiments, the side branch balloon 250 and the main branch balloon 260 may be expanded and/or inflated concurrently and/or simultaneously. In some embodiments, expanding and/or inflating the side branch balloon 250 and the main branch balloon 260 concurrently and/or simultaneously may be referred to as a kiss technique or KBI (Kiss Balloon Inflation). Some procedures require multiple KBIs, for instance, a double-kiss technique, which is used in the DK crush technique. However, unlike the DK crush technique, no portion of the side branch stent 100 is “crushed”down upon itself to create multiple layers of stent scaffold.

In some embodiments, after expanding and/or inflating the side branch balloon 250 to create the lumen opening 239 through the side wall 236 of the main branch stent 230 into the lumen of the side branch stent 100, no more than a single layer of stent scaffold of the main branch stent 230 and a single layer of stent scaffold of the side branch stent 100 overlap in intimate contact and/or in direct contact with each other against and/or along the inner wall of the body lumen 10. In some embodiments, after expanding and/or inflating the side branch balloon 250 to create the lumen opening 239 through the side wall 236 of the main branch stent 230 into the lumen of the side branch stent 100, no more than a single layer of stent scaffold of the main branch stent 230 and a single layer of stent scaffold of the side branch stent 100 are stacked directly on top of each other against and/or along the inner wall of the body lumen 10. For example, only a single layer of the side wall 236 of the main branch stent 230 may be expanded against the extension portion 130 of the side branch stent 100. Likewise, only the side wall of the extension portion 130 of the side branch stent 100 may be pressed against the side wall 236 of the main branch stent 230. One advantage of the instantly disclosed stent system and method over the DK crush technique is that fewer layers of stent scaffold are stacked on top of each other against and/or along the inner wall of the body lumen 10. Additional advantages are also contemplated and/or will be apparent to the skilled artisan. When treating Coronary Artery Disease, fewer layers of stent scaffold will facilitate the endothelization of the scaffold which will reduce endothelial shear stress and risk of adverse cardiac events (i.e., in-stent restenosis) in the long term. The eyelet 140 ensures accurate ostial positioning and minimum protrusion of the side branch stent 230 into the main branch. Another advantage is a reduction in procedural time (less steps are required to complete the stenting of the vessel) and in patient's discomfort during the intervention. Also, it reduces the complexity of the 2-stent strategy, simplifying the procedure, and ultimately permitting a larger range of physicians being able to perform the technique.

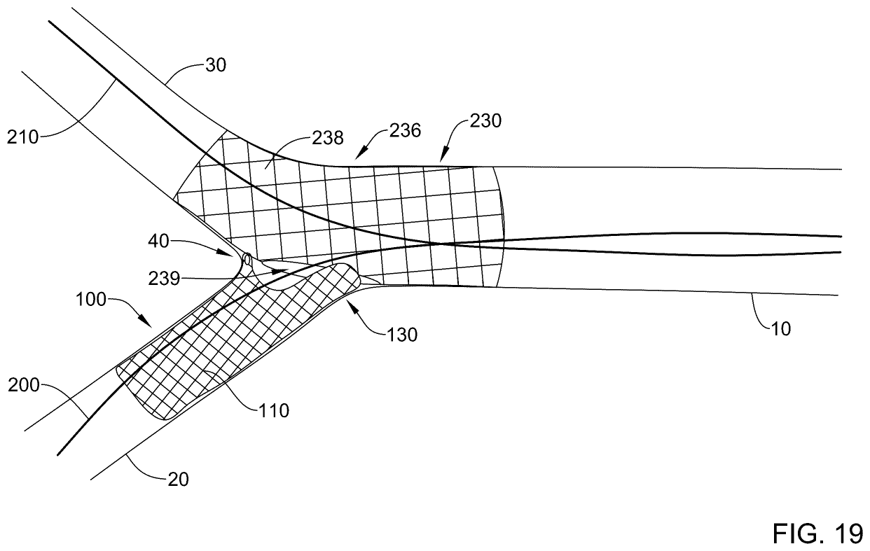

In some embodiments, the method may comprise collapsing and/or deflating the side branch balloon 250 toward and/or to the deflated configuration and collapsing and/or deflating the main branch balloon 260 toward and/or to the deflated configuration. Subsequently thereafter, the method may comprise retracting and/or removing the side branch balloon 250 from the lumen of the side branch stent 100 and/or the body lumen 10, and retracting and/or removing the main branch balloon 260 from the lumen of the main branch stent 230 and/or the body lumen 10, as seen in FIG. 19.

In some embodiments, the method may comprise, during and/or after retracting and/or removing the side branch balloon 250 and the main branch balloon 260 from the body lumen 10, maintaining the first guidewire 200 in position within the body lumen 10 and the lumen of the side branch stent 100 and maintaining the second guidewire 210 in position within the body lumen 10 and the lumen of the main branch stent 230, as seen in FIGS. 19-20. In some embodiments (though not expressly illustrated), the method may comprise, after retracting and/or removing the side branch balloon 250 and the main branch balloon 260 from the body lumen 10, retracting and/or removing the first guidewire 200 from the body lumen 10. In some embodiments (though not expressly illustrated), the method may comprise, after retracting and/or removing the side branch balloon 250 and the main branch balloon 260 from the body lumen 10, retracting and/or removing the second guidewire 210 from the body lumen 10. In some embodiments (though not expressly illustrated), the method may comprise, after retracting and/or removing the side branch balloon 250 and the main branch balloon 260 from the body lumen 10, retracting and/or removing the first guidewire 200 or the second guidewire 210 (e.g., one of the guidewires, but not both guidewires) from the body lumen 10. In some alternative embodiments (though not expressly illustrated), the method may comprise, after retracting and/or removing the side branch balloon 250 and the main branch balloon 260 from the body lumen 10, retracting and/or removing the first guidewire 200 and the second guidewire 210 from the body lumen 10 and subsequently advancing a different guidewire into the body lumen 10. In some embodiments, a distal end of the different guidewire may be further advanced into either the side branch 20 or the main branch 30.

In some embodiments, the method may comprise, after retracting and/or removing the side branch balloon 250 and the main branch balloon 260 from the body lumen 10, advancing the POT balloon 270 into the body lumen 10 and/or into the lumen of the main branch stent 230 proximal of the bifurcation 40 in a deflated configuration such that the POT balloon 270 is disposed within the proximal portion of the main branch stent 230 proximal of the bifurcation 40, as seen in FIG. 20. In some embodiments, the POT balloon 270 may be advanced into the body lumen 10 and/or into the lumen of the main branch stent 230 in the deflated configuration over the second guidewire 210, as shown in FIG. 20. In some embodiments, the POT balloon 270 may be advanced into the body lumen 10 and/or into the lumen of the main branch stent 230 in the deflated configuration over the first guidewire 200. In some alternative embodiments, the POT balloon 270 may be advanced into the body lumen 10 and/or into the lumen of the main branch stent 230 in the deflated configuration over a different guidewire.

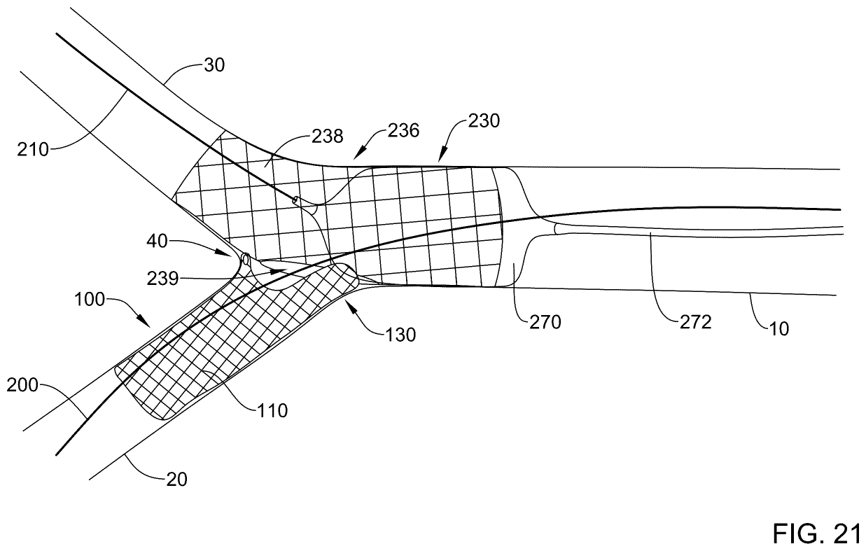

Thereafter, the POT balloon 270 may be expanded and/or inflated to an inflated configuration within the proximal portion of the main branch stent 230 to seat or re-seat the main branch stent 230 against the inner wall of the body lumen 10, as seen in FIG. 21. In some embodiments, the POT balloon 270 may have an outer diameter in the inflated configuration that is greater than an outer diameter of the second delivery balloon 240 in the inflated configuration. Seating or re-seating the proximal portion of the main branch stent 230 with the POT balloon 270 may further urge the main branch stent 230 into engagement and/or contact with the inner wall of the body lumen 10, which may improve anchoring characteristics, migration characteristics, etc. compared to initial deployment with the second delivery balloon 240. In some embodiments, expanding and/or inflating the POT balloon 270 to the inflated configuration within the proximal portion of the main branch stent 230 may improve engagement between the inner surface of the extension portion 130 of the side branch stent 100 and the outer surface 238 of the main branch stent 230 and/or the side wall 236 of the main branch stent 230. In some embodiments, expanding and/or inflating the POT balloon 270 to the inflated configuration within the proximal portion of the main branch stent 230 may reduce any residual spacing between the inner surface of the extension portion 130 of the side branch stent 100 and the outer surface 238 of the main branch stent 230 and/or the side wall 236 of the main branch stent 230. In some embodiments, expanding and/or inflating the POT balloon 270 to the inflated configuration within the proximal portion of the main branch stent 230 may improve endothelization characteristics of the first layer of stent scaffold and the second layer of stent scaffold where the inner surface of the extension portion 130 of the side branch stent 100 engages and/or contacts the outer surface 238 of the main branch stent 230 and/or the side wall 236 of the main branch stent 230.

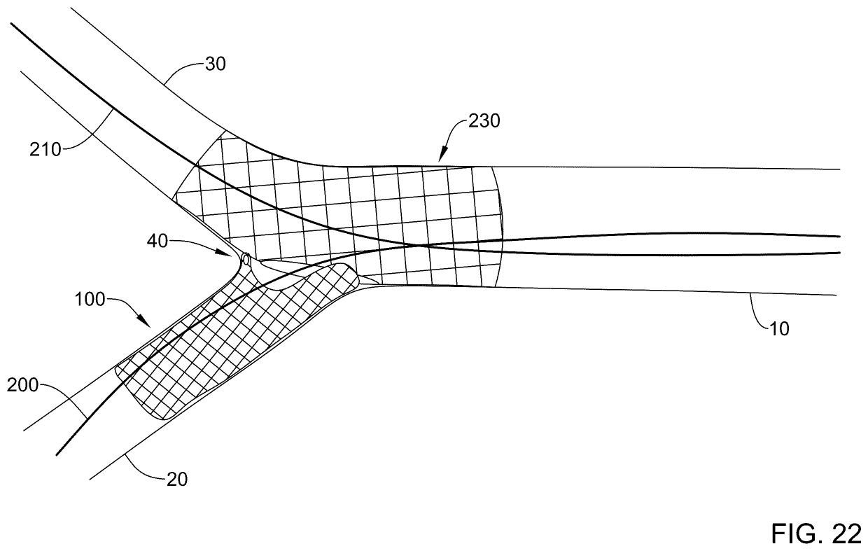

In some embodiments, the method may comprise deflating the POT balloon 270 toward and/or to the deflated configuration, and removing the POT balloon 270 from the body lumen 10, as seen in FIG. 22. In some embodiments (though not expressly illustrated), the method may comprise removing any and/or all remaining guidewires from the body lumen 10 (unless further intervention or treatment is being provided). In some embodiments (though not expressly illustrated), the method may comprise removing the first guidewire 200 from the body lumen 10. In some embodiments (though not expressly illustrated), the method may comprise removing the second guidewire 210 from the body lumen 10. In some embodiments (though not expressly illustrated), the method may comprise removing the first guidewire 200 and the second guidewire 210 from the body lumen 10.

In human anatomy, there are many instances of a body lumen that bifurcates in branches (i.e., biliary tree, pulmonary tree, coronary tree, peripheral vasculature of limbs, etc.). The disclosed stent system may be suitable to treat not only Coronary Artery Disease but also other conditions where precise and accurate scaffolding of ostial lesions is required. The disclosed stent system and associated method reduces procedural times and avoids crushing stents (creating 3-layers of struts). Also, the disclosed stent system and associated method minimizes the protrusion of the side branch stent into the main branch. It may also reduce the complexity of techniques like DK-crush (used for Coronary Artery Disease) allowing for a greater number of physicians to master the 2-stent technique.

The materials that can be used for the various components of the endoprosthesis and/or the endoprosthesis system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the guidewires, the side branch stent, the main branch stent, the delivery balloons, the side branch and main branch balloons, the POT balloon, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, bioabsorbable polymers (for example, poly-l-lactic acid (PLLA), poly lactic-co-glycolic acid (PLGA), etc.), other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A side branch stent for supporting a side branch of a body lumen at a bifurcation, comprising:
a main body portion having a proximal end, a distal end, a lumen extending therethrough, and an outer surface defining a circumference;
an extension portion extending proximally from the proximal end of the main body portion, the extension portion extending circumferentially around less than a full circumference of the main body portion; and
an eyelet extending proximally from the proximal end of the main body portion circumferentially opposite the extension portion.

2. The side branch stent of claim 1, wherein the eyelet extends from the main body portion at an oblique angle relative to a central longitudinal axis of the main body portion.

3. The side branch stent of claim 2, wherein the eyelet extends radially outward from the circumference of the main body portion.

4. The side branch stent of claim 1, wherein the eyelet is sized and configured to slidably receive a guidewire therethrough.

5. The side branch stent of claim 1, wherein the extension portion extends circumferentially between 135 degrees and 230 degrees around the circumference of the main body portion.

6. The side branch stent of claim 5, wherein the extension portion extends circumferentially between 150 degrees and 210 degrees around the circumference of the main body portion.

7. The side branch stent of claim 1, wherein the extension portion extends circumferentially 180 degrees or less around the circumference of the main body portion.

8. The side branch stent of claim 1, wherein the extension portion comprises at least one circumferential row of undulating struts disposed proximal of the proximal end of the main body portion and extending circumferentially around less than the full circumference of the main body portion.

9. A stent system for supporting a main branch and a side branch of a body lumen at a bifurcation, comprising:
a main branch stent; and
a side branch stent comprising a main body portion having a proximal end, a distal end, a lumen extending therethrough, and an outer surface defining a circumference, an extension portion extending proximally from the proximal end of the main body portion circumferentially around less than a full circumference of the main body portion, and an eyelet extending proximally from the proximal end of the main body portion circumferentially opposite the extension portion;
wherein the side branch stent is configured to engage with a side wall of the main branch stent such that the main branch stent supports the main branch and the side branch stent supports the side branch of the body lumen at the bifurcation.

10. The stent system of claim 9, wherein the proximal end of the main body portion is configured to engage with the side wall of the main branch stent.

11. The stent system of claim 9, wherein the extension portion is configured to engage with and overlap a portion of the side wall of the main branch stent when the side branch stent is engaged with the side wall of the main branch stent.

12. The stent system of claim 9, wherein the main branch stent is formed from a first layer of stent scaffold and the side branch stent is formed from a second layer of stent scaffold, and no more than two layers of stent scaffold overlap at any given location along inner walls of the main branch, the side branch, and the body lumen.

13. A method of supporting a body lumen at a bifurcation, comprising:
positioning a first guidewire extending within the body lumen into a side branch of the body lumen at the bifurcation;
positioning a second guidewire extending within the body lumen across the bifurcation into a main branch of the body lumen distal of the bifurcation;
advancing a side branch stent over the first guidewire into the side branch of the body lumen adjacent the bifurcation on a first delivery balloon in a collapsed configuration, the side branch stent comprising a main body portion having a proximal end, a distal end, a lumen extending therethrough, and an outer surface defining a circumference, an extension portion extending proximally from the proximal end of the main body portion circumferentially around less than a full circumference of the main body portion, and an eyelet extending proximally from the proximal end of the main body portion circumferentially opposite the extension portion;
wherein the first delivery balloon is advanced over the first guidewire into the side branch while the second guidewire is positioned through the eyelet and extends into the main branch distal of the bifurcation; and
expanding the first delivery balloon within the side branch to shift the side branch stent to a deployed configuration, wherein the side branch stent engages an inner wall of the side branch and the extension portion extends into the body lumen in the deployed configuration.

14. The method of claim 13, wherein advancing the side branch stent over the first guidewire into the side branch of the body lumen adjacent the bifurcation on the first delivery balloon in the collapsed configuration self-orients the side branch stent such that the eyelet is disposed immediately adjacent the main branch at the bifurcation and the extension portion is disposed substantially opposite the main branch adjacent the body lumen.

15. The method of claim 13, further comprising:
after shifting the side branch stent to the deployed configuration, deploying a main branch stent within the body lumen and the main branch across the bifurcation by expanding a second delivery balloon advanced over the second guidewire.

16. The method of claim 15, wherein deploying the main branch stent comprises:
retracting the second guidewire out of the eyelet;
thereafter, advancing the second guidewire into the main branch distal of the bifurcation outside of the eyelet;
advancing the main branch stent partially into the main branch on the second delivery balloon in a collapsed configuration over the second guidewire such that the main branch stent extends from the body lumen across the bifurcation into the main branch; and
expanding the second delivery balloon to shift the main branch stent to a deployed configuration, wherein in the deployed configuration the main branch stent engages an inner wall of the main branch and an inner wall of the body lumen.

17. The method of claim 16, wherein in the deployed configuration of the main branch stent and the deployed configuration of the side branch stent, a convex outer surface of the main branch stent is engaged with a concave inner surface of the extension portion of the side branch stent.

18. The method of claim 15, comprising:
after deploying the main branch stent, advancing a side branch balloon through a side wall of the main branch stent into the lumen of the side branch stent in a deflated configuration such that a proximal portion of the side branch balloon is disposed within the main branch stent and a distal portion of the side branch balloon is disposed within the side branch stent, and
inflating the side branch balloon to an inflated configuration thereby creating a lumen opening through the side wall of the main branch stent into the lumen of the side branch stent.

19. The method of claim 18, wherein after expanding the side branch balloon to create the lumen opening through the side wall of the main branch stent into the lumen of the side branch stent, no more than a single layer of stent scaffold of the main branch stent and a single layer of stent scaffold of the side branch stent overlap in intimate contact with each other.

20. The method of claim 13, further comprising:
prior to deploying the side branch stent, deflecting the eyelet radially outward away from a central longitudinal axis of the main body portion such that the eyelet extends radially outward of the main body portion.
